(19) United States
(12) Patent Application Publication
(10) Pub. No.: US 2016/0265024 A1
(43) Pub. Date: Sep. 15, 2016

(54) LACTATE DEHYDROGENASE DETECTOR

(71) Applicant: EUMED BIOTECHNOLOGY CO., LTD., New Taipei City (TW)

(72) Inventors: Yen-Yuan SHEN, New Taipei City (TW); Chi-Yang PENG, New Taipei City (TW); Tzer-Ming CHEN, New Taipei City (TW)

(21) Appl. No.: 14/642,499

(22) Filed: Mar. 9, 2015

(51) Int. Cl. C12Q 1/32; G01N 27/26

(52) U.S. Cl. CPC C12Q 1/32; G01N 27/26; G01N 2333/904

(57) ABSTRACT

A lactate dehydrogenase detector, which is used to detect a content of lactate dehydrogenase in a biological sample, comprises a substrate, an electrode assembly, a hydrophobic insulation layer, a cover and a reagent-containing membrane. The electrode assembly is disposed on the substrate and including a working electrode and a reference electrode. The hydrophobic insulation layer is arranged over the substrate and has a notch. The cover hoods the notch and cooperates with the substrate to form a sample channel accommodating the reagent-containing membrane. The reagent-containing membrane is made of materials selected from a group consisting of a lactic acid, nicotinamide adenine dinucleotide (NAD), diaphorase, and potassium ferricyanide. A bioelectrochemical reaction between the reagent-containing membrane and the lactate dehydrogenase induces an electrical signal variation in the electrode assembly, which indicates the content of the lactate dehydrogenase.

### FIELD OF THE INVENTION

The present invention relates to a proteinase detector, particularly to a lactate dehydrogenase detector.

### BACKGROUND OF THE INVENTION

Lactate dehydrogenases are enzymes for conversion of lactate and pyruvate. Lactate dehydrogenases are found extensively in body tissues and include five types, i.e. LDH1, LDH2, LDH3, LDH4 and LDH5, which respectively exist in different concentrations in different tissues and thus have tissue specificities. For example, the heart, kidney and red blood cells of the human body have higher concentrations of LDH1 and LDH2; the skeletal muscles and liver of the human body have higher concentrations of LDH4 and LDH5; the lung, spleen, pancreas, thyroid, adrenal and lymph nodes of the human body have higher concentrations of LDH3. Therefore, the tissue specificities of different type lactate dehydrogenases can be used as preliminary indexes to diagnose diseases. According to researches, while a human or an animal suffers infection, the concentrations of lactate dehydrogenases in the body will rise. For example, while a human suffers hepatitis, acute liver cell injury or acute skeletal muscle injury, the concentration of LDH5 will rise; while a human suffers acute viral myocarditis, kidney necrosis or hemolytic anemia, the concentration of LDH1 will rise. While a milk cow or a sheep suffers subclinical mastitis, i.e. the animal has mastitis without any symptom being detected visually or tactilely, the concentrations of lactate dehydrogenases in the milk will rise. Therefore, the concentrations of lactate dehydrogenases in the milk can be used as preliminary indexes to diagnose mastitis of milk cows or a sheep.

Recently, there have been miniature detectors able to detect lactate dehydrogenases fast and easily. For example, a US patent publication No. 2014/0273007A1 disclosed a lactate dehydrogenase detector for examining the extraction fluid from human breasts. The detector comprises a detection plate including a sample chamber and a least one groove connected with the sample chamber. The groove has a reaction membrane for detecting lactate dehydrogenases. The reaction membrane has a fixing agent containing a lactate dehydrogenase-specific fixing antibody and a color developing agent. While a sample of a human breast extraction fluid containing lactate dehydrogenases is added to the sample chamber, the sample will flow from the sample chamber to the groove connected with the chamber. Next, the lactate dehydrogenases of the sample combine with the fixing agent to form a complex. Next, the complex reacts with the color developing agent to present a color. Then, the user can judge the concentration of lactate dehydrogenases according to the shade of the color. The abovementioned prior art is addressed to human beings. The mastitis of milk cows or sheep is normally detected via examining the milk or whey with a cytological detection method, a bacteria cultivation method or an electrophoresis method.

In the abovementioned detector for detecting lactate dehydrogenases of human beings, the content of lactate dehydrogenases is judged with naked eyes according to the shade of the color. Therefore the prior lacks accuracy. The abovementioned methods for detecting mastitis of milk cows or sheep, i.e. the cytological detection method, the bacteria cultivation method or the electrophoresis method, need larger instruments, and the processes thereof are timing-consuming.

### SUMMARY OF THE INVENTION

The primary objective of the present invention is to solve the problem: the conventional lactate dehydrogenase detectors lack sufficient accuracy.

To achieve the abovementioned objective, the present invention proposes a lactate dehydrogenase detector, which is used to detect the content of lactate dehydrogenases in a biological sample, and which comprises a substrate, an electrode assembly, a hydrophobic insulation layer, a cover and a reagent-containing membrane. The electrode assembly is disposed on the substrate and includes a working electrode, a reference electrode arranged opposite the working electrode, a measurement member, and a connection section connected with the measurement member. The hydrophobic insulation layer is arranged over the substrate and covers the measurement member and the connection section of the electrode assembly. The hydrophobic insulation layer has a notch exposing the measurement member. The cover hoods the notch and cooperates with the substrate to form a sample channel. The sample channel includes a sample taking opening extended to a circumference of the substrate and a sample blocking end opposite the sample taking opening and blocked by the hydrophobic insulation layer. The measurement member is disposed between the sample taking opening and the sample blocking end. The reagent-containing membrane is disposed inside the sample channel and made of materials selected from a group consisting of a lactic acid, nicotinamide adenine dinucleotide (NAD), diaphorase, and potassium ferricyanide. After the reagent-containing membrane contacts the lactate dehydrogenase in the biological sample, bioelectrochemical reactions take place between the reagent-containing membrane and the lactate dehydrogenase and induce an electrical signal variation in the electrode assembly, which indicates the content of the lactate dehydrogenase in the biological sample.

The present invention is characterized in using the bioelectrochemical reactions between the reagent-containing membrane and lactate dehydrogenases to detect and quantify the content of the lactate dehydrogenases, whereby to inspect related diseases. The present invention adopts a reagent-containing membrane able to detect lactate dehydrogenases in the samples taken from human bodies and animal bodies and thus has more applications.

### BRIEF DESCRIPTION OF THE DRAWINGS

FIG. 1 is a perspective view schematically showing the appearance of a lactate dehydrogenase detector according to a first embodiment of the present invention.

FIG. 2 is an exploded view schematically showing the structure of the lactate dehydrogenase detector according to the first embodiment of the present invention.

FIG. 3 is a perspective view schematically showing the appearance of a lactate dehydrogenase detector according to a second embodiment of the present invention.

FIG. 4 is an exploded view schematically showing the structure of the lactate dehydrogenase detector according to the second embodiment of the present invention.

FIG. 5 is a top view schematically showing the lactate dehydrogenase detector according to one embodiment of the present invention.

### DETAILED DESCRIPTION OF THE PREFERRED EMBODIMENTS

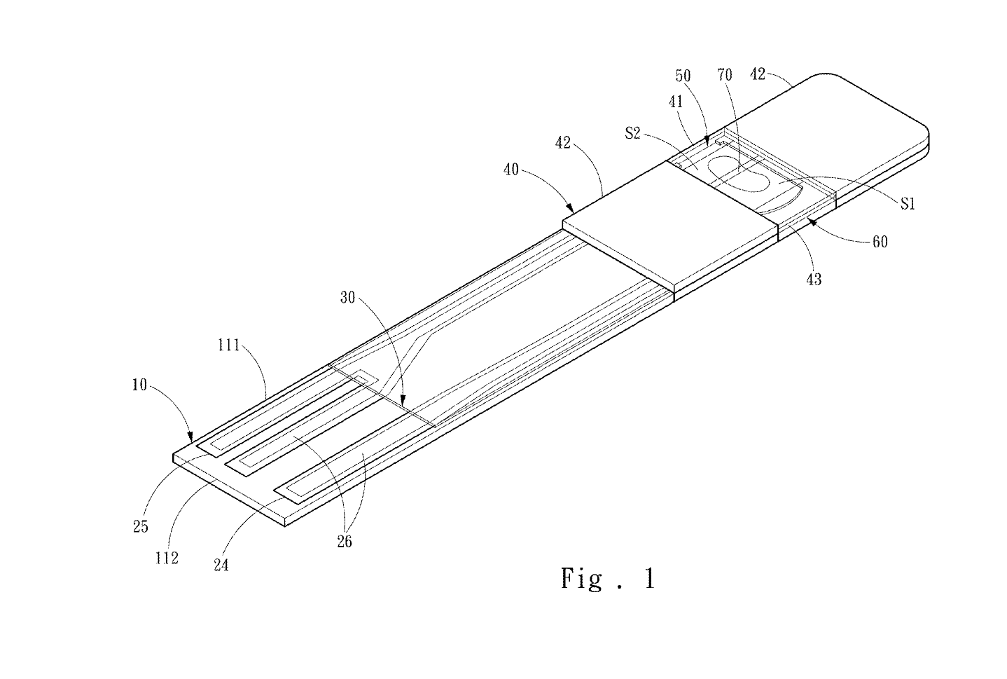

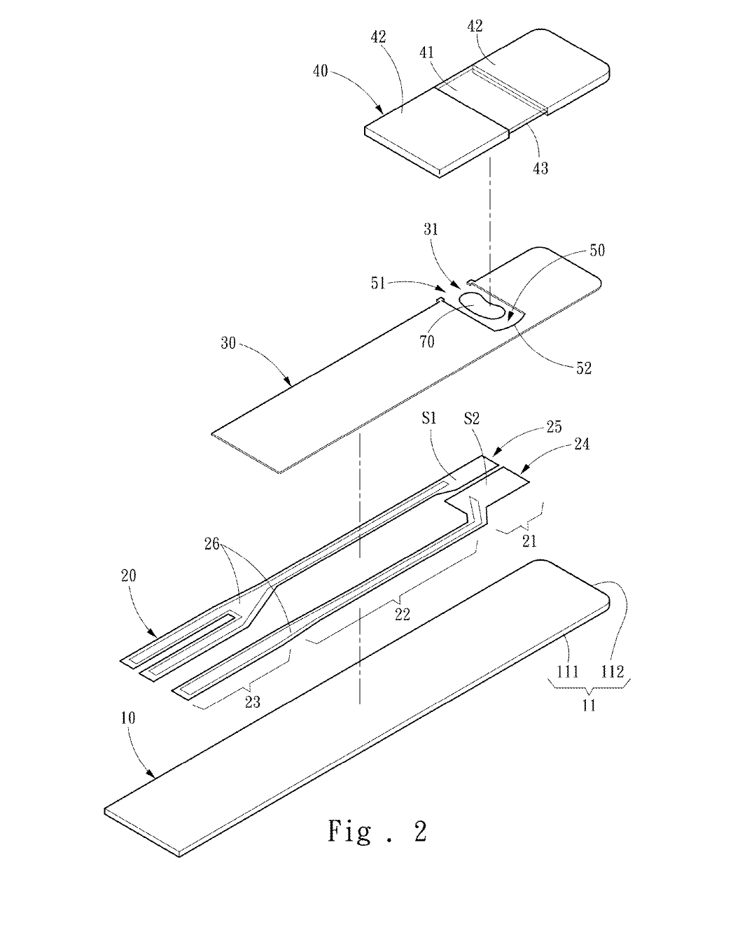

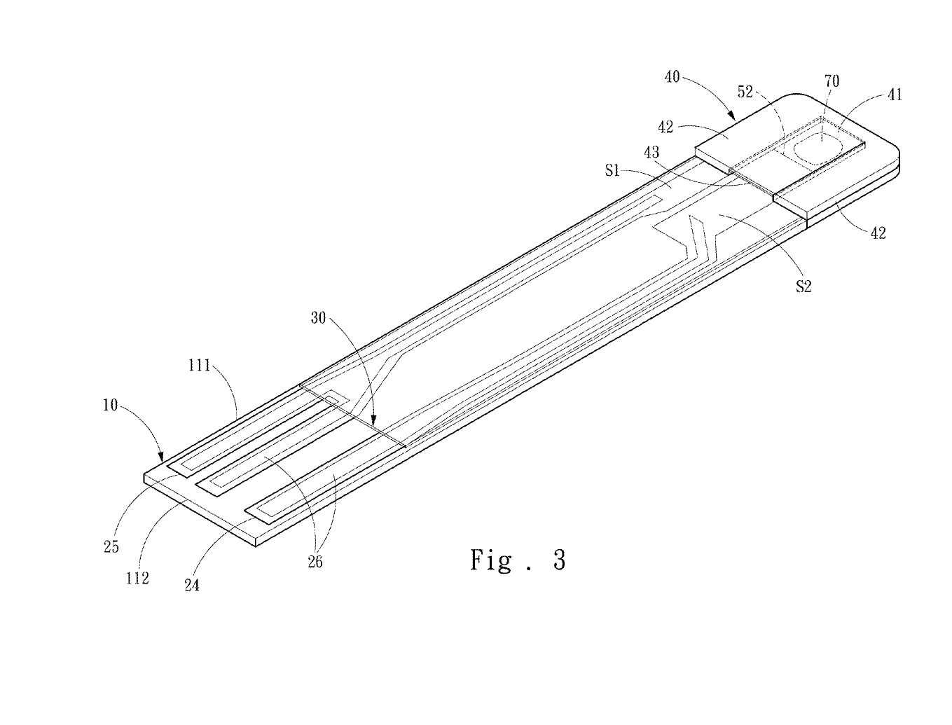

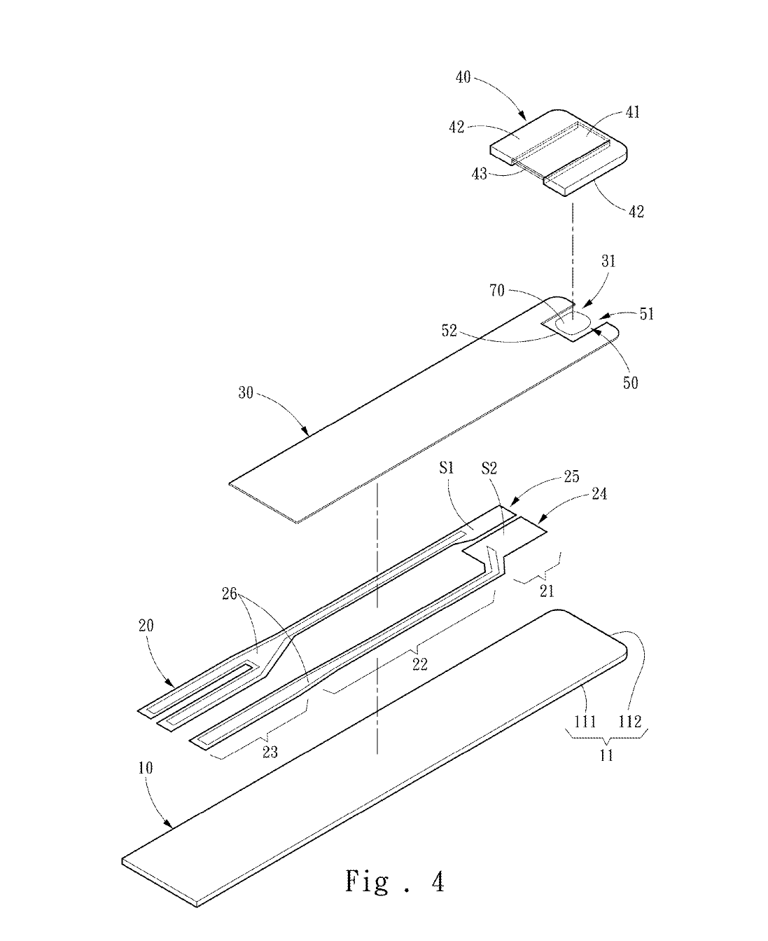

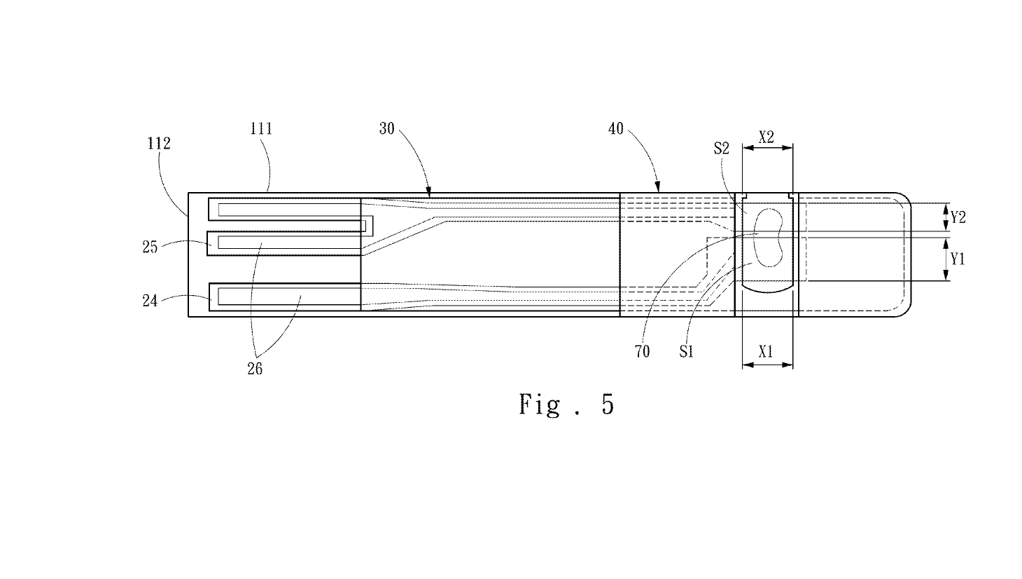

The technical contents of the present invention will be described in detail below. Refer to FIGS. 1-5. FIG. 1 is a perspective view schematically showing the appearance of a lactate dehydrogenase detector according to a first embodiment of the present invention. FIG. 2 is an exploded view schematically showing the structure of the lactate dehydrogenase detector according to the first embodiment of the present invention. FIG. 3 is a perspective view schematically showing the appearance of a lactate dehydrogenase detector according to a second embodiment of the present invention. FIG. 4 is an exploded view schematically showing the structure of the lactate dehydrogenase detector according to the second embodiment of the present invention. FIG. 5 is a top view schematically showing the lactate dehydrogenase detector according to the first embodiment of the present invention. The lactate dehydrogenase detector of the present invention is used to detect the content of the lactate dehydrogenase in a biological sample. In the first and second embodiments of the present invention, the lactate dehydrogenase detector comprises a substrate 10, an electrode assembly 20, a hydrophobic insulation layer 30 and a cover 40. In the first and second embodiments, the substrate 10 has a strip-like shape and includes a circumference 11 with two long edges 111 and two short edges 112. The two long edges 111 are parallel; the two short edges 112 are parallel and connected with the long edges 111. The electrode assembly 20 is disposed on the substrate 10 and includes a measurement member 21, a connection section 22, a signal reading member 23, a working electrode 24 and a reference electrode 25 arranged opposite the working electrode 24. The measurement member 21 and the signal reading member 23 are respectively disposed at two ends of the connection section 22. In the first and second embodiments, the connection section 22 and the signal reading member 23 are coated with an auxiliary electrode 26. In the first and second embodiments, the electrode assembly 20 is made of a conductive polymeric material, and the auxiliary electrode 26 is made of a silver paste.

The hydrophobic insulation layer 30 is made of a hydrophobic material (such as polypropylene) in form of a coating material, a laminate board or a gel. The hydrophobic insulation layer 30 is arranged over the substrate 10 and covers the measurement member 21 and connection section 22 of the electrode assembly 20. The hydrophobic insulation layer 30 has a notch 31 exposing the measurement member 21. The notch 31 is extended to one of the long edges 111 of the substrate 10 in the first embodiment, (as shown in FIG. 2) or extended to one of the short edges 112 of the substrate 11 in the second embodiment, (as shown in FIG. 4). However, the present invention does not limit that the notch 31 must be extended to the long edge 111 or the short edge 112.

The cover 40 hoods the notch 31 and cooperates with the substrate 10 to form a sample channel 50. The sample channel 50 includes a sample taking opening 51 and a sample blocking end 52. Similar to the notch 31, the sample taking opening 51 is extended to one of the long edges 111 of the substrate 10 in the first embodiment or extended to one of the short edges 112 in the second embodiment. The sample blocking end 52 is opposite the sample taking opening 51 and blocked by the hydrophobic insulation layer 30. The measurement member 21 is disposed between the sample taking opening 51 and the sample blocking end 52.

The cover 40 includes a transparent portion 41, two uplifting portions 42 and a hydrophilic layer 43. The transparent portion 41 of the cover 40 is corresponding to the notch 31 and covers the notch 31 to form the sample channel 50. The uplifting portions 42 of the cover 40 are corresponding to two sides of the notch 31 respectively and connected with the hydrophobic insulation layer 30. The uplifting portions 42 have a greater thickness than the transparent portion 41, preventing the transparent portion 41 from touching the hydrophobic insulation layer 30, and cooperating with the sample blocking end 52 to form a pocket 60 below the cover 40. The hydrophilic layer 43 is disposed on one side of the transparent portion 41, which faces the sample channel 50. The hydrophilic layer 43 is formed via coating a hydrophilic material on the surface of the transparent portion 41. The hydrophilic material is selected from a group consisting of ethyl cellulose, methyl cellulose, hydroxypropyl cellulose, cellulose acetate, polyvinyl pyrrolidone, polysulfone, polyvinylidene fluoride, polyamide, and polyimide. In one embodiment, the cover 40 is directly fabricated with a hydrophilic material and free of the hydrophilic layer 43.

In the present invention, the lactate dehydrogenase detector further comprises a reagent-containing membrane 70. The reagent-containing membrane 70 is disposed inside the sample channel 50 and contacts the working electrode 24 and the reference electrode 25. The main component of the reagent-containing membrane 70 is a lactic acid able to detect the lactate dehydrogenase in a biological sample, such as the L-lactic acid, the D-lactic acid, a lactate or a derivative thereof. In one embodiment, the reagent-containing membrane 70 includes the L-lactic acid, nicotinamide adenine dinucleotide (NAD), diaphorase, and potassium ferricyanide. In the present invention, the bioelectrochemical reaction between the reagent-containing membrane 70 and the lactate dehydrogenase of a sample, which is taken from a human or an animal, is used to detect the content of the lactate dehydrogenase. The bioelectrochemical reaction takes place in the electrode assembly 20 and causes the electrode assembly 20 to generate an electric signal variation. The electric signal variation can be measured by a measurement instrument (not shown in the drawings) electrically connected with the signal reading member 23. Then, the electric signal variation is analyzed quantitatively to determine the content of the lactate dehydrogenase.

In practical operation, the user let a biological sample flow through the sample taking opening 51 into the sample channel 50. Owing to the packet 60 and the hydrophilic layer 43, the biological sample will flow into the sample channel 50 fast. Owing to the hydrophobic insulation layer 30, the biological sample would not diffuse to the edges of the sample channel 50 but will move concentratively to the measurement member 21 inside the sample channel 50. Thereby, the biological sample will contact the reagent-containing membrane 70, the working electrode 24 and the reference electrode 25 by sufficient areas to facilitate the measurement of the content of the lactate dehydrogenase.

In one embodiment, after the biological sample flows through the sample taking opening 51 into the sample channel 50, the following bioelectrochemical reactions take place:

Lactate+NAD⁺→NADH+Pyruvate

NADH+Potassium Ferricyanide→NAD⁺+Potassium Ferrocyanide

Potassium Ferrocyanide→Potassium Ferricyanide+e⁻

wherein the lactate dehydrogenase in the biological sample catalyzes the lactate in the reagent-containing membrane 70 and converts the lactate into a pyruvate; the lactate dehydrogenase also reduces the NAD+ in the reagent-containing membrane 70 into NADH; the diaphorase in the reagent-containing membrane 70 uses NADH to catalyze potassium ferricyanide and reduce the potassium ferricyanide into potassium ferrocyanide; the potassium ferrocyanide flows to the measurement member 21; at this time, a voltage of 0.2-0.6V is applied to the working electrode 24 and the reference electrode 25 to make the potassium ferrocyanide undertake a bioelectrochemical reaction, whereby is generated a current in the measurement member 21. An instrument is electrically connected with the lactate dehydrogenase detector to measure the current. The value of the current is proportional to the concentration of the potassium ferrocyanide. Then, the concentration of the lactate dehydrogenase in the biological sample is indirectly quantified according to the value of the current.

The lactate dehydrogenase may have different types. In one embodiment, a pre-treatment process is undertaken to separate the lactate dehydrogenases. The pre-treatment process is a heat treatment process, a chromatographic process or an immunoprecipitation process, whereby to partially separate the different types of lactate dehydrogenases in the biological sample. In heat-treating a biological sample containing five types of lactate dehydrogenases, the biological sample is heated at a temperature of 60° C. for 30 minutes, and LDH4 and LDH5 will lose their enzymic activities because LDH4 and LDH5 are thermolabile. Thus, only LDH1, LDH2 and LDH3 are detected in the test. In the chromatographic process, the biological sample is passed through a microbore column for ion exchange, whereby LDH3, LDH4 and LDH5 are firstly separated, next LDH2, and lastly LDH1. In the immunoprecipitation process, the antibodies respectively specific to different types of lactate dehydrogenases are used to separate the different types of lactate dehydrogenases in sequence. Via the heat treatment process, the chromatographic process or the immunoprecipitation process, the contents of different types of lactate dehydrogenases can be respectively detected and used to determine whether the contents thereof meets a pathognomonic feature. In one embodiment, the lactate dehydrogenases are processed with electrophoresis to acquire the individual contents of the different types of lactate dehydrogenases.

Refer to FIG. 5. In one embodiment, the measurement member 21 of the working electrode 24 has a first exposed area (S1) of 4-6 mm². For example, in the sample channel 50, the measurement member 21 of the working electrode 24 is designed to have a rectangular first exposed area with a 1.8-2.2 mm first short edge Y1 and a 2.3-2.7 mm first long edge X1. In the sample channel 50, the measurement member 21 of the reference electrode 25 has a second exposed area S2 of 2.5-4.5 mm². For example, in the sample channel 50, the measurement member 21 of the reference electrode 25 is designed to have a rectangular second exposed area S2 with a 1.1-2.6 mm second short edge Y2 and a 2.3-2.7 mm second long edge X2. Thereby, the biological sample contacts the measurement member 21 of the working electrode 24 and the reference electrode 25 by such an area that facilitates the measurement of the contents of lactate dehydrogenases in the biological sample.

In conclusion, the present invention discloses a lactate dehydrogenase detector characterized in using the bioelectrochemical reactions between the reagent-containing membrane and lactate dehydrogenases to detect and quantify the content of the lactate dehydrogenases, whereby to inspect related diseases. The present invention adopts a reagent-containing membrane able to detect lactate dehydrogenases in the samples taken from human bodies and animal bodies and thus applies to more fields. For example, the present invention can detect whether a milk cow or a sheep suffers mastitis. Besides, the lactate dehydrogenase detector features compactness and portability and thus conveniences users to undertake inspections. Therefore, the present invention possesses utility, novelty and non-obviousness and meets the condition for a patent. Thus, the Inventors file the application for a patent. It is appreciated if the patent is approved fast.

The present invention has been fully demonstrated with the embodiments above. However, these embodiments are only to exemplify the present invention but not to limit the scope of the present invention. Any equivalent modification or variation according to the spirit of the present invention is to be also included within the scope of the present invention.

What is claimed is:

1. A lactate dehydrogenase detector, which is used to detect a content of lactate dehydrogenase in a biological sample, comprising
a substrate;
an electrode assembly disposed on the substrate and including a working electrode, a reference electrode arranged opposite the working electrode, a measurement member and a connection section connected with the measurement member;
a hydrophobic insulation layer arranged over the substrate, covering the measurement member and connection section, and having a notch exposing the measurement member;
a cover hooding the notch and cooperating with the substrate to form a sample channel, wherein the sample channel includes a sample taking opening extended to a circumference of the substrate and a sample blocking end opposite the sample taking opening and blocked by the hydrophobic insulation layer, and wherein the measurement member is disposed between the sample taking opening and the sample blocking end; and
a reagent-containing membrane disposed inside the sample channel and contacting the working electrode and the reference electrode, wherein the reagent-containing membrane is made of materials selected from a group consisting of a lactic acid, nicotinamide adenine dinucleotide (NAD), diaphorase, and potassium ferricyanide, and wherein after the reagent-containing membrane contacts the lactate dehydrogenase in the biological sample, a bioelectrochemical reaction takes place between the reagent-containing membrane and the lactate dehydrogenase and induces an electrical signal variation in the electrode assembly, which indicates the content of the lactate dehydrogenase in the biological sample.

2. The lactate dehydrogenase detector according to claim 1, wherein the substrate has a strip-like shape, and the circumference of the substrate includes two opposite long edges and two short edges connected with the long edges.

3. The lactate dehydrogenase detector according to claim 2, wherein the notch and the sample taking opening are disposed along the long edge.

4. The lactate dehydrogenase detector according to claim 2, wherein the notch and the sample taking opening are disposed along the short edge.

5. The lactate dehydrogenase detector according to claim 1, wherein the cover includes a transparent portion disposed above the sample channel.

6. The lactate dehydrogenase detector according to claim 5, wherein the cover includes a hydrophilic layer disposed on one side of the transparent portion, which faces the sample channel.
